(19) United States
(12) Patent Application Publication
(10) Pub. No.: US 2013/0115664 A1
(43) Pub. Date: May 9, 2013

(54) PROCESS AND SYSTEM FOR PRODUCING ALGAL OIL

(71) Applicants: Surajit Khanna, Feasterville, PA (US); Howard L. Bobb, Berlin, NJ (US)

(72) Inventors: Surajit Khanna, Feasterville, PA (US); Howard L. Bobb, Berlin, NJ (US)

(73) Assignee: BARD HOLDING, INC., Morrisville, PA (US)

(21) Appl. No.: 13/513,365

(22) PCT Filed: Dec. 3, 2010
(86) PCT No.: PCT/US10/58901

§ 371 Date: Jan. 22, 2013

Related U.S. Application Data
(60) Provisional application No. 61/266,267, filed on Dec. 3, 2009.

(51) Int. Cl. C12M 1/00

(52) U.S. Cl. CPC C12M 21/02
USPC 435/134; 435/292.1

(57) ABSTRACT

A method for producing an algal oil is provided. The method includes continuously providing a growth medium and an algal strain to a bioreactor at a predetermined fluid flow rate; illuminating the growth medium and algal strain contained within the bioreactor by a first artificial light source for a time sufficient to effect lipid production by the algal strain; continuously withdrawing a portion of the growth medium and algal strain contained within the bioreactor at the predetermined fluid flow rate; and treating the withdrawn portion of the growth medium and algal strain to produce and isolate a lipid produced by the algal strain.

### CROSS-REFERENCE TO RELATED APPLICATIONS

This application claims the benefit of U.S. Provisional Patent Application No. 61/266,267, filed Dec. 3, 2009, the entire contents of which are incorporated by reference herein.

### BACKGROUND OF THE INVENTION

The present invention relates to the production of biofuels and, more particularly, to the production of algal-based biofuels. Biofuels can be obtained or produced from various vegetable feedstocks and are useful as an alternative to fossil fuel. Soybeans, palm, and corn, for example, are considered to be the first generation of biofuels feedstock. Soybeans are grown in the United States and provide a good ratio of oil production per acre when compared to other types of vegetables, such as corn, as feedstock. However, there are certain disadvantages of using soybeans as a biofuel feedstock. One such disadvantage is the variability in the cost of soybeans and, in particular, the risk of extreme spikes in cost, as occurred during the years 2008-2009. One catalyst for potential spikes in the cost of soybeans is the competition that exists for soybeans to be used as both fuel and food. Another disadvantage of using these types of feedstocks for the production of oil is that these types of feedstocks require a significant amount of land for the production of the feedstock, and such land could instead be used for the production of food crops. The negative impact of clearing of rain forests around the globe for the cultivation of vegetable oils is well documented. Therefore, it is desirable to produce substantial quantities of biofuels without these adverse effects.

Algae has been recognized as a potential source of oil to convert into biofuels. Algae is a fast growing microorganism that contains high percentages of lipids. These lipids can be harvested and converted into biofuels. The primary process for algae production has conventionally been the use of open ponds which rely on natural sunlight to provide the necessary photons for algae growth. However, conventional open pond technology faces various challenges, such as maintaining temperature control, preventing contamination, evaporation, limitations of the diurnal cycle, and the requirement of significant amounts of land. Open ponds also suffer a particular disadvantage, in that they do not provide a controlled environment for optimal algae growth. Also, conventional open ponds are relatively shallow in depth, because sunlight can only penetrate the algae to a limited extent, such that the conventional open ponds require a large surface area of land.

Other conventional algae growth systems involve the growing of algae in tubes that allow sunlight to pass through the outer walls of the tubes to stimulate growth, much as the sun would stimulate algal growth in an open pond. The tubes are generally positioned horizontally which allows for some positive product management, but which minimizes the output per acre yield of the growth system. Nevertheless, because algae multiplies autonomously and can be cultivated using raw materials having relatively low cost (or, potentially, negative cost, in that algae can consume solid, liquid, and gaseous waste products, thereby avoiding disposal costs), the potential of algal production of biofuel products remains tantalizing.

Accordingly, it is desirable to provide a method for producing algal-based fuel which overcomes some of the economic barriers associated with vegetable feedstocks and the environmental control difficulties associated with conventional algal-based fuel production processes.

Algae grows without human intervention almost everywhere on the planet that there is moisture and sunlight. The process described herein is intended to enhance the growth and potential harvest of algae oil, relative to natural or open-pond growth of algae.

### BRIEF SUMMARY OF THE INVENTION

Briefly stated, in one embodiment, the present invention is directed to a method for producing an algal oil. The method includes continuously providing a growth medium and an algal strain to a bioreactor at a predetermined fluid flow rate; illuminating the growth medium and algal strain contained within the bioreactor by a first artificial light source for a time sufficient to effect lipid production by the algal strain; continuously withdrawing a portion of the growth medium and algal strain contained within the bioreactor at the predetermined fluid flow rate; and treating the withdrawn portion of the growth medium and algal strain to produce and isolate a lipid produced by the algal strain.

According to another embodiment, the present invention is directed a method for producing an algal oil including pre-incubating the algal strain in an incubation tank; continuously providing nutrients to the incubation tank; continuously withdrawing a portion of the nutrients and algal strain from the incubation tank at the predetermined fluid flow rate; continuously providing the withdrawn portion of the nutrients and algal strain, to a carbonation tank at the predetermined fluid flow rate and providing carbon dioxide to the carbonation tank to form a growth medium; continuously providing the growth medium and algal strain to a plurality of substantially vertically oriented bioreactors at the predetermined-fluid flow rate; illuminating the growth medium and algal strain contained within the bioreactors by an artificial light source for a time sufficient to effect lipid production by the algal strain; continuously withdrawing a portion of the growth medium and algal strain contained within the bioreactors at the predetermined fluid flow rate; and treating the withdrawn portion of the growth medium and algal strain to produce and isolate a lipid produced by the algal strain.

According to another embodiment, the present invention is directed to a system for producing an algal oil. The system includes a plurality of bioreactors configured to continuously receive a growth medium and an algal strain at a predetermined fluid flow rate and to continuously output a portion of the growth medium and the algal strain at the predetermined fluid flow rate, wherein each of plurality of bioreactors comprises an artificial light source comprising a blue light at a wavelength of 420 to 450 nanometers and a red light at a wavelength of 640 to 680 nanometers, wherein the artificial light source has an illuminance of 2,500 to 10,000 lux.

### BRIEF DESCRIPTION OF THE SEVERAL VIEWS OF THE DRAWINGS

The foregoing summary, as well as the following detailed description of the invention, will be better understood when read in conjunction with the appended drawing. For the purpose of illustrating the invention, there are shown in the drawing embodiments which are presently preferred. It should be understood, however, that the invention is not limited to the precise arrangements and instrumentalities shown.

In the drawing:

FIG. 1 is a schematic block diagram illustrating a process for producing algal-based biofuel according to preferred embodiment of the present invention.

### DETAILED DESCRIPTION OF THE INVENTION

The present invention relates to a system and method for producing products that may be utilized as fuels from cultivated algae. It will be understood by those skilled in the art that the products produced from the below described process may be utilized for various other purposes. More particularly, the present invention relates to a method for producing an algal oil.

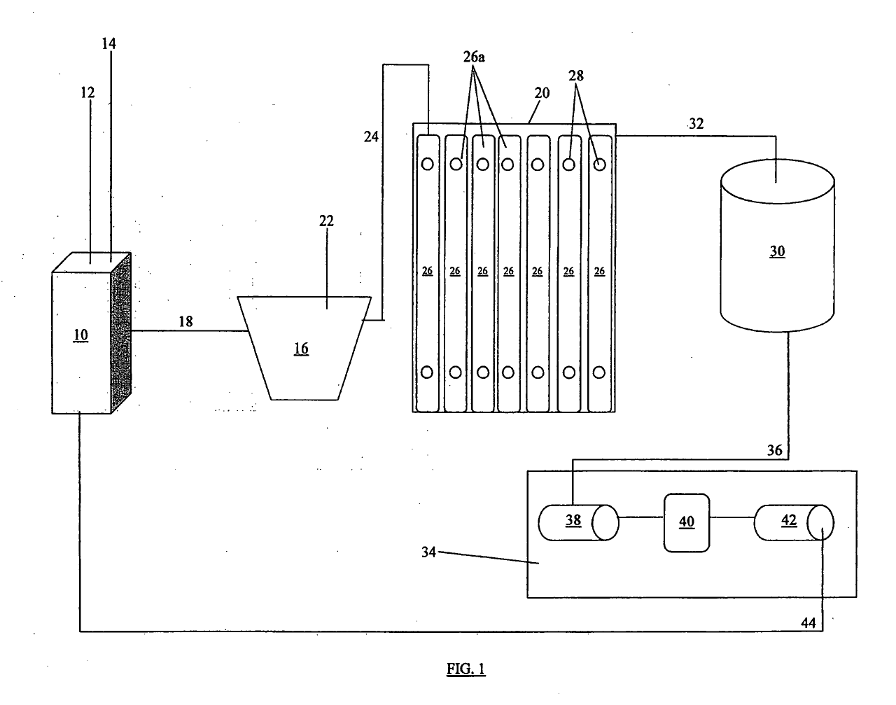

The method comprises combining carbon dioxide, water and nutrients required for lipid production by an algal strain to form a growth medium. The ratio of carbon dioxide to water is between approximately 50-100 cubic feet per hour per 1,000 gallons of water per day. Approximately 3.75 liters of combined nutrients are provided on a daily basis. Specifically, referring to FIG. 1, the process begins with the formation of a growth medium for an algae strain in an incubation tank 10. Water and the nutrients required for growth of the algae are provided to the incubation tank 10 at a flow rate of approximately 0.1 to 10 gallons per minute. More preferably, the water and nutrients are provided to the incubation tank 10 at a flow rate of approximately 1 gallon per minute. Preferably, water is fed or pumped to the incubation tank 10 via a first conduit 12 and the nutrients are fed or pumped to the incubation tank 10 via a second conduit 14.

Examples of the nutrients provided to the incubation tank 10 for generation of the growth medium or system include, but are not limited to, nitrogen, phosphorus, potassium, silica and iron. However, it will be understood by those skilled in the art that any nutrients suitable for algae growth may be used.

The water to be combined with nutrients may be sourced from a variety of resources, such as potable dechlorinated water, primary waste water and secondary waste water. Preferably, the water is wastewater because wastewater is readily available and relatively inexpensive, such that the commercial potential for algae production is dramatically increased. Also, common municipal wastewater contains nitrates which, if handled properly, greatly enhances the growth potential of algae. Thus, the wastewater itself may serve as the sole or an additional nutrient source for generation of the growth medium. The wastewater, however, must be monitored periodically and, more preferably, continuously to ensure that the acidic potential of nitric acid contained in the wastewater is never reached, as such wastewater would be very harmful to the algae seeds. This is preferably accomplished by monitoring the pH balance of the wastewater being added to the incubation tank 10. An alkaline salt, such as magnesium bicarbonate is also added to the incubation tank 10 to maintain the appropriate pH level of the wastewater. The expense associated with monitoring the wastewater is generally offset by the elimination of or reduced need for and costs associated with the municipality treating the wastewater prior to disposal thereof.

Preferably, the nutrients are continuously delivered to the incubation tank 10 via an automated delivery system connected to one or more probes for monitoring, for example, the pH, temperature and conductivity of the contents of the incubation tank 10. It will be understood by those skilled in the art, however, that the nutrients may be fed to the incubation tank 10 by any appropriate delivery system or mechanism.

In one embodiment, a starter culture of the algae resides in the incubation tank 10 and receives the water and nutrients. The components are then subjected to incubation and a portion of the growth medium and algal strain are continuously withdrawn from the incubation tank at a predetermined fluid flow rate. The growth medium and algal strain may be withdrawn at the predetermined fluid flow rate of approximately 0.1 to 10 gallons per minute, preferably 0.1 to 5 gallons per minute, or more preferably 0.1 to 3 gallons per minute. Most preferably, however, the growth medium and algal strain are withdrawn at the predetermined fluid flow rate of approximately 1 gallon per minute. Specifically, once the algae reach maturity in the incubation tank 10, the mixture of the mature algae, water and nutrients is continuously withdrawn from the incubation tank at the predetermined fluid flow rate and fed or pumped to a carbonation tank 16 via a third conduit 18 at the predetermined fluid flow rate. Typically, the algae may take from approximately 24 to 48 hours to reach maturity in the incubation tank 10. However, not all of the algae contained within the incubation tank 10 need to reach maturity. Instead, as some of the algae reach maturity, the mature algae will naturally float to the top of the incubation tank 10, such that only the mature algae may be skimmed and withdrawn from the incubation tank 10 and preferably continuously fed to the carbonation tank 16 along with the water and nutrients. Thus, a continuous flow mode is achieved at the incubation stage of the process. In another embodiment, the algae may reside in the cultivation module 20 described herein.

Also, in the incubation tank 10, the water, nutrients and algal strain are subjected to illumination by an artificial light source. Preferably, the contents of the incubation tank 10 are illuminated by an artificial light source comprising a blue light source and a red light source. The artificial blue light preferably has a wavelength of 420 to 450 nanometers and, more preferably, a wavelength of 435 nanometers, and a light intensity suitable for the growth of algae. The artificial red light preferably has a wavelength of 640 to 680 nanometers and, more preferably, a wavelength of 658 nanometers, and a light intensity suitable for the growth of algae. Preferably, the artificial light source has an illuminance of 2,500 to 10,000 lux and, more preferably, 3,000 lux.

Preferably, the carbon dioxide is continuously fed to the carbonation tank 16 and is obtained from a combustion exhaust which is the result of a power generation process. For example, a coal fired power generating plant may be utilized as the source of the carbon dioxide feed. Preferably, this is accomplished by locating the system adjacent or proximate to a coal fired power generating plant. Emitters such as coal fired power generating plants have, under regulation, installed scrubbers to clean their emissions in order to limit the content of harmful chemicals. However, in addition to carbon dioxide, coal fired power generating plants may inevitably still emit sulfur, mercury or other chemicals which could harm or stunt the growth of algae. Thus, the carbon dioxide stream which is diverted or captured from such emitters may periodically be retested to determine if further scrubbing is necessary. While such retesting of the carbon dioxide stream is an added expense for the production process, the expense is generally offset by the increased growth potential of the algae, as well as by the sale of the clean oxygen which can be exhausted from the algae growth system and marketed, for example, to health and commercial industries.

Accordingly, an aqueous growth medium containing sufficient nutrients and carbon dioxide to support algal life, proliferation, and oil (lipid) production is obtained. The growth medium and algal strain to be cultivated are then fed or otherwise provided a cultivation module 20 via a fifth conduit 24. The cultivation module 20 comprises a cultivation tube or bioreactor 26 and, more preferably a plurality of bioreactors 26, for active and continuous growth of the algae. The growth medium and the algal strain are preferably continuously provided to the plurality of bioreactors 26 at the predetermined fluid flow rate. Accordingly, a continuous and rapid growth mode is achieved.

The pH of the feed stream (represented by the fifth conduit 24) of the growth medium and algae is preferably continuously monitored. More preferably, prior to providing the growth medium to the bioreactors 26, the pH of the growth medium is adjusted to and maintained at a pH suitable for growth of the algal strain. Preferably, the pH of the growth medium is adjusted to and maintained at a pH of from approximately 8 to approximately 11.5. More preferably, the pH of the growth medium is adjusted to and maintained at a pH of 8.5.

Preferably, the bioreactors 26 are clustered together in a module design. Each of the plurality of bioreactors 26 is preferably oriented in a substantially vertical position, such that the longitudinal axis of each bioreactor 26 is generally perpendicular to the surface on which the bioreactor 26 is situated. The substantially vertical orientation of the bioreactors 26 facilitates algal cultivation at a relatively high yield per acre, particularly since the volume of bioreactors 26 per acre of land is substantially increased relative to conventional bioreactors 26. The bioreactors 26 are also preferably substantially tubular in form.

The modular design of the bioreactors 26 supports scalability. Each module is preferably composed of 7 bioreactors. Each, bioreactor 26 has a height of approximately eight to ten feet and a diameter of approximately 23 to 28 inches. Preferably, each bioreactor 26 has a height of approximately 8 feet and a diameter of approximately 23.5 inches. By carefully controlling the identity of the algal strain used and the growth conditions, cultivation times of as little as approximately every 5 hours or less may be achieved. Examples of the algal strains that may be utilized include, but are not limited to, chlamydomonas reindardtii, chlorella vulgaris, chlorella pyrenoidosa, and ochromonas danica.

Each of the plurality of bioreactors 26 preferably includes an artificial light source to illuminate the contents of the bioreactors 26. Preferably, the contents of the bioreactors 26 are illuminated by an artificial light source comprising a blue light source and a red light source. The artificial blue light preferably has a wavelength of 420 to 450 nanometers and, more preferably, a wavelength of 435 nanometers, and a light intensity suitable for the growth of algae. The artificial red light preferably has a wavelength of 640 to 680 nanometers and, more preferably, a wavelength of 658 nanometers, and a light intensity suitable for the growth of algae. Preferably, the artificial light source has an illuminance of 2,500 to 10,000 lux and; more preferably, 8,000 lux. The necessary light intensity will vary based on the algal strain utilized. Light is a necessary component for successful growth of algae seeds and production of algal oil. Algae grows freely in sunlight, but does not substantially grow in continuous darkness. The infusion of a light source at the proper wavelength keeps the algae in a permanent growth cycle. The artificial light according to the present invention keeps the algae in a constant growth phase minimizing the anaerobic digestion which occurs during dark periods.

In one embodiment, at least one artificial light source (not shown) is disposed within the lumen of the tubular form of the bioreactor 26. The artificial light sources are disposed within the interior 26a, and preferably the center, of each of the bioreactors 26 to provide a continuously-available light source to supply light to algae growing within the bioreactors 26. Thus, continuous or intermittent algal growth maybe promoted based upon user specifications. Preferably, the contents of the bioreactors 26 are illuminated by the light sources for a time sufficient to effect lipid or oil, production by the algal strain. The algae begins the growth process as soon as it is exposed to light and is subjected to either continuous light or intermittent light depending on targeted growth rates.

Preferably, the internal artificial light sources are substantially tubular in shape and extend substantially the entire height of the bioreactors 26. More preferably, the internal artificial light sources are light-emitting diodes (LEDs). A secondary and external source of light is also provided by external light assemblies (not shown) mounted on at least a portion of the outside of each of the bioreactors 26. Preferably, the external artificial light sources extend substantially the entire height of the bioreactors 26. Preferably, each bioreactor is provided with apertures 28, such as portholes 28, which serve as access points for the external lighting to continuously or intermittently supply light to the Algae growing within the bioreactor. The sources of artificial light thus supplant the need for sunlight for growth of the algae.

The external surface of the internal light source and the internal surface of the bioreactor 26 define the bounds of the algal growth system within each bioreactor 26. Fouling of the lights sources and the bioreactors 26 by adherence of growing algae to either the external surface of the internal light sources or the internal surface of the bioreactor 26 could dramatically shield the light rays of the internal and external light sources from penetrating the algae culture and slow the growth cycle. Continuous or intermittent cleaning of the external surfaces of the internal light sources and the internal surfaces of the bioreactors 26 helps to reduce algae interference with illumination and maintain the desired light intensity. The cleaning may be accomplished by, for example, wiping, scraping, abrading, or otherwise dislodge algae from these surfaces using brushes or other physical displacement devices. For example, brushes which are actuated using a timed and motorized mechanism may traverse the full length of the interior of each of the bioreactors 26 at predetermined intervals to eliminate loss of light and enhance growth potential. More preferably, specifically fashioned brushes are attached to pulleys by a cable system and are mechanically pulled up and down inside each of the bioreactors 26 at predetermined intervals to clean the light sources and bioreactors 26. The cleaning preferably occurs on a weekly basis.

For continuous algal growth, a source of light is necessary only 12.5% to 14.29% of the time. However, because an artificial light source is utilized, the contents of the bioreactors can be selectively illuminated only during the necessary durations, in order to maximize growth while minimizing energy usage. Furthermore, generation of light preferentially at wavelengths used by the algae can further limit energy consumption attributable to illumination activities. Preferably, the bioreactors 26 are made of polyvinyl chloride (PVC). Also, preferably, at least a portion of the interior surface of each of the plurality of bioreactors 26 has reflective properties, such that light within the interior of each bioreactor 26 is reflected throughout the interior of the bioreactor 26 to maximize the effect of the internal light source to its best potential. More preferably, the entire internal surface of each bioreactor 26 is reflective.

The cultivation phase of the process is continued until a desired quantity of algae and/or oil are produced. Cultivation times are determined empirically and vary, depending on numerous factors within the control of the operator including, for example, the identity of the algal strain, the composition of the growth medium, the composition of the carbon, dioxide feed stream, the pH of the growth medium, the temperature of the growth medium, the light intensity within the bioreactors 26, and the initial culture density of the algal strain. Preferably, the cultivated algae is approximately doubled ten to twelve over on a daily basis.

Following growth of the algae within the bioreactors 26, a portion of the contents of each of the bioreactors 26 is withdrawn or harvested from the bioreactors 26. In particular, because of the substantially vertical configuration of the bioreactors 26, mature algae which has been cultivated can easily float to the top of the bioreactors 26, and is continuously harvested such that other algae contained within the bioreactors which has not yet reached maturity gains sufficient exposure to the light source for cultivation. Thus, a portion of the growth medium and algal strain (i.e., the cultivated and mature algae) contained within the bioreactors, is continuously withdrawn from the bioreactors 26 at the predetermined fluid flow rate. Preferably, approximately 50% of the contents of the bioreactors 26 are removed from the bioreactors 26.

The harvested or withdrawn algae are then sent to a harvesting tank 30 via a sixth conduit 32. The harvesting activity is performed in an upflow mode, analogous to the flow within the substantially vertically-oriented bioreactors 26. Specifically, the algae is harvested via a gravity flow system. As the algae rises up the length of the vertical column of the bioreactor 26, the algae flows over and is funneled to the harvesting tank 30. Harvesting of a portion of the contents of the bioreactors 26 may be performed on a continuous basis until the desired percentage (i.e., 50%) of the contents are removed. Alternatively, the desired percentage (i.e., 50%) of the contents may be removed all at once. The algae remaining in the bioreactors 26 continue to grow, multiply and refill the bioreactors 26 as additional enriched water and carbon dioxide are introduced into the system from the carbonation tank 16.

The withdrawn portion of the growth medium and algal strain is then treated to produce and isolate a lipid. Specifically, algae contained in the withdrawn or harvested portion are then sent to an extraction system 34 via a seventh conduit 36, In the extraction system 34, the withdrawn or harvested portion of fluid is subjected to a treatment to separate the algae mass from water contained therein. Preferably, the harvested fluid is sent through a centrifuge 38 for separation of the water from the algae mass. The algae mass is then subjected to a treatment to isolate the lipid or algal oil from the treated portion of algal cells. Preferably, the algae mass of the withdrawn portion is subjected to ultrasonic treatment using an industrial ultrasonic processor 40 sufficient to rupture at least some cells of the algal strain to release the algae. However, other treatments, such as chemical solvent extraction and mechanical crushing, may also be utilized.

The resulting product, made up of the algal oil and the algal cells, is then subjected to a further treatment, such as flotation, sedimentation or centrifugation, for separation of the algal oil from the algal cells. Preferably, the algal oil and algal cell are subjected to centrifugation in a centrifuge 42 for separation of the algal oil from the algal cells. Any excess water and carbon dioxide are then re-circulated to the incubation tank 10 via a recirculation conduit 44 preferably at a fluid flow rate of 0.5 gallons per minute.

The resulting algal oil is then processed to produce a biofuel. Preferably, the algal oil undergoes transesterification to produce glycerol and biodiesel. The resulting biodiesel is a mixture of long-chain fatty acid esters that can be used, either alone or mixed with a petroleum-derived diesel fuel, for various purposes, such as a fuel in diesel engines. The remaining mass of algal cells, which is composed of proteins and carbohydrates, is dried and pressed into algae cake. Other products that are made in the process and can be collected include oxygen, which is a byproduct of algal photosynthesis. The algae cake, in particular, is an algal solids product that can be used as animal feed, fish feed, nutritional supplements for human and animal consumption, fertilizer, a dry fuel, or for other purposes.

The equipment used in this process can be arranged very compactly due to various characteristics. In particular, because the bioreactor tubes are arranged in a substantially vertical orientation, the bioreactors 26 can be installed within a relatively small geometric footprint, such as within excess space at a power-generating facility or a municipal waste water plant. Since two of the core inputs for the algae growth process are carbon dioxide and water, co-location of the required equipment at sites such as power-generating facilities or a municipal waste water plants is particularly advantageous. In addition, the bioreactors 26 can be stacked vertically to take full advantage of available space. Also, because the bioreactors 26 contain artificial light sources required for algal growth, exposure to ambient sunlight is not required. Also, since the design is based on a modular concept, there is flexibility in terms of production capacity, based on available space. The compactness, scalability, and, modularity of the process and equipment described herein render the process suitable for installation in a wide variety of settings, particularly including settings in which algal nutrient (e.g., sewage or other waste water) and/or carbon dioxide streams are economically available.

Growth of algae requires carbon dioxide, water, algae seed, and either sunlight or an alternative light source. Enhancing the growth of algae to increase the economic potential can be achieved by calibrating each of the aforementioned components by a combination of processes which individually contributes to algal production but, employed in combination, synergistically enhance algal production significantly.

It will be appreciated by those skilled in the art that changes could be made lo the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for producing an algal oil, the method comprising:
continuously providing a growth medium and an algal strain to a bioreactor at a predetermined fluid flow rate;
illuminating the growth medium and algal strain contained within the bioreactor by a first artificial light source for a time sufficient to effect lipid production by the algal strain;
continuously withdrawing a portion of the growth medium and algal strain contained within the bioreactor at the predetermined fluid flow rate; and
treating the withdrawn portion of the growth medium and algal strain to produce and isolate a lipid produced by the algal strain.

2. The method of claim 1, wherein the predetermined fluid flow rate is one gallon per minute.

3. The method of claim 1, wherein the growth medium and algal strain are provided to a plurality of bioreactors.

4. The method of claim 1, wherein the growth medium and algal strain are provided to a substantially vertically-oriented bioreactor.

5. The method of claim 1, wherein the first artificial light source comprises a blue light at a wavelength of 420 to 450 nanometers and a red light at a wavelength of 640 to 680 nanometers, wherein the first artificial light source has an illuminance of 2,500 to 10,000 lux.

6. The method of claim 5, wherein the first artificial light source comprises a blue light at a wavelength 435 nanometers and a red light at a wavelength of 658 nanometers and has an illuminance of 8,000 lux.

7. The method of claim 1, further comprising pre-incubating the algal strain in an incubation tank illuminated by a second artificial light source; wherein nutrients are continuously provided to the incubation tank and the algal strain is continuously withdrawn from the incubation tank at the predetermined fluid flow rate.

8. The method of claim 7, wherein the second artificial light source comprises a blue light at a wavelength of 420 to 450 nanometers and a red light at a wavelength of 640 to 680 nanometers and an illuminance of 2,500 to 10,000 lux.

9. The method of claim 8, wherein the second artificial light source comprises a blue light at a wavelength of 435 nanometers and a red light at a wavelength of 658 nanometers and has an illuminance of 3,000 lux.

10. The method of claim 7, further comprising continuously providing the algal strain withdrawn from the incubation tank to a carbonation tank, continuously providing carbon dioxide to the carbonation tank, continuously withdrawing the growth medium and the algal strain from the carbonation tank and providing the growth medium and the algal strain to the bioreactor, wherein all of the streams are provided and withdrawn at the predetermined fluid flow rate.

11. The method of claim 1, further comprising adjusting the pH of the growth medium prior to providing the growth medium to the bioreactor to a value suitable for growth of the algal strain.

12. A method for producing an algal, oil, the method comprising:
pre-incubating the algal strain in an incubation tank;
continuously providing nutrients to the incubation tank;
continuously withdrawing a portion of the nutrients and algal strain from the incubation tank at the predetermined fluid flow rate;
continuously providing the withdrawn portion of the nutrients and algal strain to a carbonation tank at the predetermined fluid flow rate and providing carbon dioxide to the carbonation tank to form a growth medium;
continuously providing the growth medium and algal strain to a plurality of substantially vertically oriented bioreactors at the predetermined fluid flow rate;
illuminating the growth medium and algal strain contained within the bioreactors by an artificial light source for a time sufficient to effect lipid production by the algal strain;
continuously withdrawing a portion of the growth medium and algal strain contained within the bioreactors at the predetermined fluid flow rate; and
treating the withdrawn portion of the growth medium and algal strain to produce and isolate a lipid produced by the algal strain.

13. A system for producing an algal oil comprising a plurality of bioreactors configured to continuously receive a growth medium and an algal strain at a predetermined fluid flow rate and to continuously output a portion of the growth medium and the algal strain at the predetermined fluid flow rate, wherein each of plurality of bioreactors comprises an artificial light source comprising a blue light at a wavelength of 420 to 450 nanometers and a red light at a wavelength of 640 to 680 nanometers, wherein the artificial light source has an illuminance of 2,500 to 10,000 lux.

14. The system of claim 13, wherein each of plurality of bioreactors comprises an artificial light source comprising a blue light at a wavelength of 435 nanometers and a red light at a wavelength of 658 nanometers and the artificial light source has an illuminance of 8,000 lux.

15. The system of claim 13, wherein the plurality of bioreactors are substantially vertically oriented.
